SEQUENCE LISTING (16 sequences)

SEQ ID NO: 1 - DNA, 32 bp, Artificial Sequence
  Description of Artificial Sequence Synthetic primer for HasA - forward
agtccatggaatacaaagcgcaagaaaggaac

SEQ ID NO: 2 - DNA, 29 bp, Artificial Sequence
  Description of Artificial Sequence Synthetic primer for HasA - reverse
atcgcatgcctcccttgtcagaacctagg

SEQ ID NO: 3 - DNA, 33 bp, Artificial Sequence
  Description of Artificial Sequence Synthetic primer for HasB - forward
gtccatggaagaaatgaaaatttctgtagcagg

SEQ ID NO: 4 - DNA, 31 bp, Artificial Sequence
  Description of Artificial Sequence Synthetic primer for HasB - reverse
atcgcatgcctagtctcttccaaagacatct

SEQ ID NO: 5 - DNA, 35 bp, Artificial Sequence
  Description of Artificial Sequence Synthetic primer for HasC - forward
gtccatggaagaactcatgacaaaggtcagaaaag

SEQ ID NO: 6 - DNA, 29 bp, Artificial Sequence
  Description of Artificial Sequence Synthetic primer for HasC - reverse
atcgcatgcgctctgcaatagctaagcca

SEQ ID NO: 7 - DNA, 35 bp, Artificial Sequence
  Description of Artificial Sequence Synthetic primer for GlmU - forward
gtccatggaaaggaatcaaaacatgaaaaactacg

SEQ ID NO: 8 - DNA, 29 bp, Artificial Sequence
  Description of Artificial Sequence Synthetic primer for GlmU - reverse
atctctagaactatagcttactggggctg

SEQ ID NO: 9 - DNA, 37 bp, Artificial Sequence
  Description of Artificial Sequence Synthetic primer for Pgi - forward
gtccatggaagggagtaaaataatgtcacatattaca

SEQ ID NO: 10 - DNA, 26 bp, Artificial Sequence
  Description of Artificial Sequence Synthetic primer for Pgi - reverse
atcgcatgcttacaagcgtgcgttga

SEQ ID NO: 11 - DNA, 29 bp, Artificial Sequence
  Description of Artificial Sequence Synthetic primer for GlmS - forward
actccatggacggtgttaagttatgtgtg

SEQ ID NO: 12 - DNA, 30 bp, Artificial Sequence
  Description of Artificial Sequence Synthetic primer for GlmS - reverse
agctctagatggcaggcaactattactcaa

SEQ ID NO: 13 - DNA, 35 bp, Artificial Sequence
  Description of Artificial Sequence Synthetic primer for PgiGlmU - forward
catctagacgaggaatcaaaacatgaaaaactacg

SEQ ID NO: 14 - DNA, 38 bp, Artificial Sequence
  Description of Artificial Sequence Synthetic primer for PgiGlmU - reverse
caaagctttatagcttactggggctgatccgggtgatg

SEQ ID NO: 15 - DNA, 26 bp, Artificial Sequence
  Description of Artificial Sequence Synthetic primer for GuaB - forward
gttgatgtggttaaggttggtatcgg

SEQ ID NO: 16 - DNA, 24 bp, Artificial Sequence
  Description of Artificial Sequence Synthetic primer for GuaB - reverse
agccttggaagtaacggtcgcttg

(19) United States
(12) Patent Application Publication
(10) Pub. No.: US 2011/0281817 A1
(43) Pub. Date: Nov. 17, 2011

(54) PRODUCTION OF HYALURONIC ACID

(75) Inventors: Lars Keld Nielsen, St. Lucia (AU); Wendy Chen, St. Lucia (AU); Esteban Stefane Marcellin Saldana, West End (AU)

(73) Assignees: SUGAR INDUSTRY INNOVATION PTY. LTD., St. Lucia (AU); THE UNIVERSITY OF QUEENSLAND, Brisbane (AU)

(21) Appl. No.: 12/675,727

(22) PCT Filed: Aug. 29, 2008
(86) PCT No.: PCT/AU08/01267

§ 371 Date: Jan. 18, 2011

(30) Foreign Application Priority Data
Aug. 31, 2007 (EP) 07115404.1
May 14, 2008 (AU) 2008902353

(51) Int. Cl. A61K 31/728; A61K 8/73; C12N 1/20; C12P 19/18; C12P 19/04; C12N 1/21; C08B 37/08; C12P 19/24

(52) U.S. Cl. 514/54; 536/53; 435/94; 435/97; 435/101; 435/252.3; 435/253.4

(57) ABSTRACT

Methods for producing hyaluronic acid are described, including altering the activity in Streptococcus cells of one or more enzymes and/or altering the amount of available substrates or substrate precursors.

### FIELD OF THE INVENTION

The present invention relates to methods for the production of hyaluronic acid in Streptococcus sp., as well as to hyaluronic acid produced by such methods.

### BACKGROUND TO THE INVENTION

Hyaluronic acid (HA) is a uniformly repetitive, linear glycosaminoglycan composed of 2,000-25,000 disaccharides of glucuronic acid and N-acetylglucosamine joined alternately by β-1-3 and β-1-4 glycosidic bonds: [β-1,4-glucuronic acid-β-1,3-N-acetyl glucosamine-]ₙ.

Reflecting its variety of natural functions, HA has found a number of applications in medicine, cosmetics and speciality foods. In many applications, high molecular weight is a desired property and different approaches have been employed to produce high molecular weight (MW) HA.

High MW HA can be obtained through careful extraction from rooster comb. HA in rooster combs may reach very high values, for instance up to 12-14 million (M) Dalton (Da). Depending on the extraction process, a final product of 3-5 MDa can be obtained (U.S. Pat. No. 4,141,973). Increased reticence to the use of animal derived products in medicine and cosmetics has seen a shift towards microbial HA production. Microbial HA production through fermentation of group C streptococci, in particular Streptococcus equi subsp. equi and S. equi subsp. zooepidemicus, has been practised commercially since the early 1980s. Microbial HA, however, is of lower molecular weight (typically 0.5 to 2 MDa) than HA obtainable from rooster comb.

In some applications, chemical cross-linking has been used to increase molecular weight (e.g., U.S. Pat. No. 4,582,865; U.S. Pat. No. 6,903,199; U.S. Pat. No. 7,125,860; and U.S. Pat. No. 6,703,444). In other applications, notably ophthalmic applications, cross-linking is undesirable and strain engineering is the only means of realising high MW HA.

HA is synthesised as an extracellular capsule by pathogenic Lancefield group A and C streptococci. Under the microscope, these non-sporulating and nonmotile bacteria appear as spherical or ovoid cells that are typically arranged in pairs or chains surrounded by an extensive extracellular capsule. On sheep blood agar plates, colonies of these β-hemolytic bacteria will produce a clear zone with HA identified as a mucoid or slimy translucent layer surrounding bacterial colonies. The HA capsule is a virulence factor in these streptococci, presumably affording the bacterium a stealth function as the immune system of higher organisms fails to recognise the HA capsule as a foreign entity

HA is produced by polymerisation of two activated glycosyl donors, UDP-glucuronic acid (UDP-GUA) and UDP-N-acetylglucosamine (UDP-NAG), in a reaction catalysed by HA synthase (EC 2.4.1.212) (FIG. 1). The two precursors are synthesised in two pathways branching from glucose-6-phosphate. The first pathway starts with the conversion of glucose-6-phosphate to glucose-1-phosphate by α-Phosphoglucomutase (EC 5.4.2.2). UDP-glucose pyrophosphorylase (EC 2.7.7.9) catalyses the reaction of UTP and glucose-1-phosphate to produce the nucleotide sugar UDP-glucose. UDP-GUA is then obtained by specific oxidation of the primary alcohol group of UDP-glucose through the action of UDP-glucose dehydrogenase (EC 1.1.1.22). The second pathway involved in the production of amino sugars starts with the conversion of glucose-6-phosphate into fructose-6-phosphate catalysed by phosphoglucoisomerase (EC 5.3.1.9). Amino group transfer from glutamine to fructose-6-phosphate by an amidotransferase (EC 2.6.1.16) yields glucosamine-6-phosphate. Phosphate group rearrangement by a mutase (EC 5.4.2.10) generates glucosamine-1-phosphate from glucosamine-6-phosphate. Acetyl group transfer by an acetyltransferase (EC 2.3.1.4) forms N-acetyl glucosamine-6-phosphate. Finally, a pyrophosphorylase (EC 2.7.7.23) adds UDP to obtain UDP-NAG.

In addition to their role in HA production, the two pathways are required for the biosynthesis of cell wall components. Intermediates in the UDP-GUA pathway are used in the biosynthesis of cell wall polysaccharides and teichoic acid. UDP-NAG is the source of amino sugars in lipopolysaccharides, proteoglycans as well as peptidoglycans. The first step in peptidoglycan synthesis is catalysed by UDP-N-Acetylglucosamine 1-carboxyvinyltransferase (UDP-NAG-CVT) (EC 2.5.1.7), which joins UDP-NAG and phosphoenolpyruvate to form UDP-N-acteyl-3-O-(1-carboxyvinyl)-glucosamine.

The HA synthase plays an important role in controlling HA MW and site directed mutagenesis has been employed to modify HA MW (Kumari, K., et al. (2006). “Mutation of Two Intramembrane Polar Residues Conserved within the Hyaluronan Synthase Family Alters Hyaluronan Product Size.” J. Biol. Chem. 281(17): 11755-11760). Random mutagenesis followed by strain selection has also been used to improve strain properties including HA MW (Kim, J.-H., et al. (1996). “Selection of a Streptococcus equi mutant and optimization of culture conditions for the production of high molecular weight hyaluronic acid.” Enzy. Microbial Tech. 19(6): 440-445; Lee, M. S., at al. (1999). “Construction and analysis of a library for random insertional mutagenesis in Streptococcus pneumoniae: Use for recovery of mutants defective in genetic transformation and for identification of essential genes.” Appl. Environ. Microbiol. 65(5): 1883-1890; U.S. Pat. No. 5,496,726; U.S. Pat. No. 7,323,329).

Several studies have demonstrated that in addition to the HA synthase (HasA) high UDP-glucose dehydrogenase activity (HasB) is required to achieve high HA yields. Expression of HasA in heterologous hosts such as Escherichia coli, Bacillus subtilis or Lactococcus lactis yields little or no HA unless HasB is overexpressed as well (DeAngelis P. polypeptide, or a variant, analogue or fragment thereof, L., et al. (1993) “Molecular cloning, identification, and sequence of the hyaluronan synthase gene from group A Streptococcus pyogenes”. J. Biol. Chem. 268:19181-19184; WO 03/054163; Chien L. J., Lee C. K. (2007) “Hyaluronic acid production by recombinant Lactococcus lactis.” Appl. Microbiol. Biotechnol. 77:339-346).

### SUMMARY OF THE INVENTION

In a first aspect, the present invention provides a method for producing hyaluronic acid which method comprises growing Streptococcus cells in a culture medium, which cells express the enzymes required for hyaluronic acid synthesis, wherein the activity or amount in the cells of one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase has been increased; and optionally recovering the hyaluronic acid produced by the cells.

In a second aspect, the present invention provides a method for producing hyaluronic acid which method comprises recovering hyaluronic acid from Streptococcus cells that express the enzymes required for hyaluronic acid synthesis, wherein the activity or amount in the cells of one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase has been increased.

In a third aspect, the present invention provides a method for producing hyaluronic acid which method comprises growing Streptococcus cells in a culture medium, which cells express the enzymes required for hyaluronic acid synthesis, wherein the cells have been engineered or treated to increase the activity or amount in the cells of one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase; and optionally recovering the hyaluronic acid produced by the cells.

In a fourth aspect, the present invention provides a method for producing hyaluronic acid which method comprises recovering hyaluronic acid from Streptococcus cells that express the enzymes required for hyaluronic acid synthesis, wherein the cells have been engineered or treated to increase the activity or amount in the cells of one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase.

In a fifth aspect, the present invention provides a method for producing hyaluronic acid which method comprises growing Streptococcus cells in a culture medium, which cells express the enzymes required for hyaluronic acid synthesis, and providing one or more substrates selected from UDP-N-acetylglucosamine, N-acetylglucosamine and glucosamine; and optionally recovering the hyaluronic acid produced by the cells.

In a sixth aspect, the present invention provides a method for producing hyaluronic acid which method comprises recovering hyaluronic acid from Streptococcus cells that express the enzymes required for hyaluronic acid synthesis, wherein one or more substrates selected from UDP-N-acetylglucosamine, N-acetylglucosamine and glucosamine has been provided.

In a seventh aspect, the present invention provides a method for producing hyaluronic acid which method comprises growing Streptococcus cells in a culture medium, which cells express the enzymes required for hyaluronic acid synthesis, wherein the cells have been engineered or treated to increase the amount in the cells of one or more substrates selected from UDP-N-acetylglucosamine, N-acetylglucosamine and glucosamine; and optionally recovering the hyaluronic acid produced by the cells.

In an eighth aspect, the present invention provides a method for producing hyaluronic acid which method comprises recovering hyaluronic acid from Streptococcus cells that express the enzymes required for hyaluronic acid synthesis, wherein the cells have been engineered or treated to increase the amount in the cells of one or more substrates selected from UDP-N-acetylglucosamine, N-acetylglucosamine and glucosamine.

In a ninth aspect, the present invention provides a method for producing hyaluronic acid which method comprises growing Streptococcus cells in a culture medium, which cells express the enzymes required for hyaluronic acid synthesis, wherein the activity or amount in the cells of one or more enzymes selected from UDP-N-acetylglucosamine 1-carboxyvinyltransferase and undecaprenyldiphospho-muramoylpentapeptide beta-N-acetylglucosaminyltransferase has been decreased or abrogated; and optionally recovering the hyaluronic acid produced by the cells.

In a tenth aspect, the present invention provides a method for producing hyaluronic acid which method comprises recovering hyaluronic acid from Streptococcus cells that express the enzymes required for hyaluronic acid synthesis, wherein the activity or amount in the cells of one or more enzymes selected from UDP-N-acetylglucosamine 1-carboxyvinyltransferase and undecaprenyldiphospho-muramoylpentapeptide beta-N-acetylglucosaminyltransferase has been decreased or abrogated.

In an eleventh aspect, the present invention provides a method for producing hyaluronic acid which method comprises growing Streptococcus cells in a culture medium, which cells express the enzymes required for hyaluronic acid synthesis, wherein the cells have been engineered or treated to decrease or abrogate the activity or amount in the cells of one or more enzymes selected from UDP-N-acetylglucosamine 1-carboxyvinyltransferase and undecaprenyldiphospho-muramoylpentapeptide beta-N-acetylglucosaminyltransferase; and optionally recovering the hyaluronic acid produced by the cells.

In a twelfth aspect, the present invention provides a method for producing hyaluronic acid which method comprises recovering hyaluronic acid from Streptococcus cells that express the enzymes required for hyaluronic acid synthesis, wherein the cells have been engineered or treated to decrease or abrogate the activity or amount in the cells of one or more enzymes selected from UDP-N-acetylglucosamine 1-carboxyvinyltransferase and undecaprenyldiphospho-muramoylpentapeptide beta-N-acetylglucosaminyltransferase.

In a thirteenth aspect, the present invention further provides hyaluronic acid obtained or obtainable by the methods of the invention. The hyaluronic acid may have an average molecular weight of at least 3 or 3.5 MDa. The hyaluronic acid may be substantially non-crosslinked.

In a fourteenth aspect, the present invention provides a Streptococcus cell which comprises the enzymes for synthesis of hyaluronic acid, which cell has been genetically modified to overexpress one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase.

In a fifteenth aspect, the present invention provides a Streptococcus cell which comprises the enzymes for synthesis of hyaluronic acid, which cell has been genetically modified to underexpress or not express or express with downregulated activity one or more enzymes selected from UDP-N-Acetylglucosamine 1-carboxyvinyltransferase and undecaprenyldiphospho-muramoylpentapeptide beta-N-acetylglucosaminyltransferase.

In a sixteenth aspect, the present invention provides a pharmaceutical composition comprising the hyaluronic acid of the present invention and a pharmaceutically acceptable carrier, excipient or diluent.

In a seventeenth aspect, the present invention provides a cosmetic composition comprising the hyaluronic acid of the present invention and a cosmetically acceptable carrier, excipient or diluent.

In an eighteenth aspect, the present invention provides a food product or food additive comprising the hyaluronic acid of the present invention.

### DEFINITIONS

Unless defined otherwise, all technical and scientific terms used herein have the same meaning as commonly understood by one of ordinary skill in the art (e.g. in cell biology, chemistry, molecular biology and cell culture). Standard techniques used for molecular and biochemical methods can be found in Sambrook et al., Molecular Cloning: A Laboratory Manual, 3rd ed. (2001) Cold Spring Harbor Laboratory Press, Cold Spring Harbor, N.Y. and Ausubel et al., Short Protocols in Molecular Biology (1999) 4th Ed, John Wiley & Sons, Inc.—and the full version entitled Current Protocols in Molecular Biology).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, reference to numerical values, unless stated otherwise, is to be taken as meaning “about” that numerical value. The term “about” is used to indicate that a value includes the inherent variation of error for the device and the method being employed to determine the value, or the variation that exists among the study subjects.

The reference to any prior art in this specification is not, and should not be taken as an acknowledgement or any form of suggestion that prior art forms part of the common general knowledge in Australia.

### BRIEF DESCRIPTION OF THE FIGURES

The present invention will now be described, by way of example only, with reference to the following figures.

FIG. 1 shows a schematic flow chart of the biosynthetic pathways leading to production of hyaluronic acid.

FIG. 2 shows a 2D gel of S. zooepidemicus (ATCC 35246) showing the location of UDP N-acetylglucosamine 1-carboxyvinyltransferase (EC 2.5.1.7) (UDP-NAG-CVT). Proteins were harvested using hyaluronidase to remove the HA capsule. Proteins were separated using pH gradient 4-7 and 24 cm 12% polycarylamide gels. Proteins were labelled with cy3 and visualised using a typhoon scanner. Protein spots were identified using LC/MS/MS and MALDI/TOF/TOF.

FIG. 3 shows stationary phase production of HA in fed batch culture for wildtype S. zooepidemicus (ATCC 35246) under anaerobic conditions (Panel A) and for S. zooepidemicus carrying a pNZ plasmid encoding for gimU and pgi (Panel B). Standard cultures were fermented to exhaustion of glucose and left for another 30 min to deplete an essential amino acid. Upon feeding of glucose, hyaluronic acid production recommenced while biomass remained constant.

### DETAILED DESCRIPTION OF THE INVENTION

The inventors have explored the effect of overexpressing enzymes involved in the biosynthesis of HA precursors in streptococci that naturally produce a high HA yield. While enhanced expression had a limited effect on HA yield, the inventors surprisingly found that enhanced expression of particular enzymes involved in the biosynthesis of HA precursors leads to an increase in the molecular weight of the HA produced.

Cells that have been engineered to express enhanced levels of enzymes involved in the UDP-NAG pathway (for example, phosphoglucoisomerase, D-fructose-6-phosphate amidotransferase, glucosamine-1-phosphate acetyl transferase and N-acetyl glucosamine-1-phosphate pyrophosphorylase) produced HA with a significantly higher molecular weight compared to wild type cells and cells that had been engineered to overexpress the HA synthase or enzymes involved in the UDP-GUA pathway (for example, UDP-glucose dehydrogenase and UDP-glucose pyrophosphorylase).

The inventors further determined that cells with elevated levels of UDP-NAG produced HA with increased molecular weight. This was true for cells overexpressing genes in the UDP-NAG pathway compared to wild type cells and cells expressing genes in the UDP-GUA pathway. It was also true for cells carrying an empty plasmid control, which were found to express higher levels of GImU (glucosamine-1-phosphate acetyl transferase/N-acetyl glucosamine-1-phosphate pyrophosphorylase), and lower levels of UDP-NAG-CVT, an enzyme catalysing the first UDP-NAG dependent step in peptidoglycan biosynthesis.

Accordingly, the inventors have concluded that the molecular weight of HA can be increased by increasing the availability of UDP-NAG, which may be achieved by increasing the activity of enzymes producing UDP-NAG, by supplementing the medium with substrates that the cell converts into UDP-NAG and/or by reducing the activity of enzymes that compete with HA synthase for UDP-NAG.

### Methods and Cells for Increasing Enzyme Expression or Activity

The present invention is partly based on the finding that increased expression/activity of a number of enzymes in the pathway for hyaluronic acid production in Streptococcus sp. leads to an increase in the molecular weight (MW) of the resulting hyaluronic acid produced by the cells. The specific enzymes identified as giving rise to an increase in HA MW are phosphoglucoisomerase (HasE, Pgi—EC 5.3.1.9), D-fructose-6-phosphate amidotransferase (GlmS—EC 2.6.1.16) and glucosamine-1-phosphate acetyl transferase/N-acetylglucosamine-1-phosphate pyrophosphorylase (HasD, GlmU—EC 2.3.1.4 and 2.7.7.23).

Thus in the methods of the invention, the streptococcus cells may have increased activity/expression of one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase.

In some embodiments, the streptococcus cells have been genetically modified to overexpress a heterologous gene, for example, a eukaryotic gene encoding glucosamine-6-phosphate acetyl transferase or phosphoacetylglucosamine mutase.

Preferably the cells have increased activity/expression of at least phosphoglucoisomerase.

In one embodiment, cells have wild type levels and activity of HA synthase (HasA).

Increased expression/activity may be measured relative to an equivalent wild-type strain which has not been genetically modified and which is grown under standard conditions (such as 37° C. in rich media (M17G) or in chemically defined media (CDM) supplemented with 2% w/v D-glucose). For example, in the case of mucoid Group C Streptococcus equi subsp. zooepidemicus, a suitable control strain is ATCC 35246.

In one embodiment, increased activity of the enzymes is effected by genetically engineering the cells by introducing one or more nucleic acid sequences that direct expression of the enzymes. Such sequences can be introduced by various techniques known to persons skilled in the art, such as the introduction of plasmid DNA into cells using electroporation followed by subsequent selection of transformed cells on selective media. These heterologous nucleic acid sequences may be maintained extrachromosomally or may be introduced into the host cell genome by homologous recombination.

Accordingly, the present invention provides a Streptococcus cell which comprises the enzymes for synthesis of hyaluronic acid, which cell has been genetically modified to overexpress one or more enzymes selected from phosphoglucoisomerase, D-fructose-6-phosphate amidotransferase, phosphoglucosamine mutase, glucosamine-1-phosphate acetyl transferase, N-acetylglucosamine-1-phosphate pyrophosphorylase, glucosamine-6-phosphate acetyl transferase, and phosphoacetylglucosamine mutase. In a particular embodiment, the cell comprises one or more heterologous nucleic acid sequences encoding one or more of the enzymes. In another embodiment, the cells comprise one or more mutations in genomic regulatory sequences encoding the one or more enzymes, which mutations result in increased levels of expression of the one or more enzymes, relative to a wild type cell. In a further embodiment, the cells may comprise one or more mutations in the coding sequences of the one or more enzymes that give rise to increased enzyme activity. Combinations of these embodiments are also possible.

It is particularly preferred in relation to the methods and cells described above, that the cells have increased activity/expression of phosphoglucoisomerase and/or glucosamine-1-phosphate acetyl transferase/N-acetylglucosamine-1-phosphate pyrophosphorylase.

Nucleic acid sequences encoding the enzymes of interest, operably linked to regulatory sequences that are capable of directing expression of the enzymes in a suitable Streptococcus host cell, can be derived from a number of sources. The HAS operons from four streptococcal species have been cloned to date. The sequence of hasD/glmU has been cloned for S. equisimilus and S. equis subsp. zooepidemicus. Further, the sequence of hasE/pgi has been cloned for S. equis subsp. zooepidemicus. Sequences can also be obtained from other species, e.g. B. subtilis has a homologue of hasB termed tuaD. HasD/gImU has been cloned for a variety of bacterial species e.g. S. pyogenes (Accession No. YP_{—}001129027); E. coli (Accession Nos. ABG71900 and P0ACC7) and B. subtilis (Accession No. P14192). The complete genomes of S. pyogenes, E. coli and B. subtilis have been sequenced and published.

By way of a further example, suitable oligonucleotide primers for amplifying hasD (glmU), hasE (pgi) and glmM sequences from S. zooepidemicus genomic DNA are described in the experimental section below.

In some embodiments, the nucleic acid sequences encoding one or more of the enzymes of interest are operably linked to regulatory sequences that are inducible so that expression of the enzymes is upregulated as desired, by the addition of an inducer molecule to the culture medium.

An alternative approach is to modify the host cell's regulatory sequences that control expression of the endogenous sequences encoding the enzymes of interest by homologous recombination, e.g. promoter sequences.

A further approach is to treat the cells such that amplification of the endogenous sequences occurs, resulting in increased copy number of the endogenous DNA encoding the enzymes of interest, leading to increased expression and activity of the enzymes.

It is also possible to subject cells to various mutagenesis treatments and to test for increases in enzyme activity using enzyme assays known in the arts, examples of which are described in the experimental section. It is also possible to use site-directed mutagenesis to modify the coding sequence of the enzymes to increase enzyme activity.

The activity of the enzymes of interest can also be upregulated using chemical treatments, e.g. molecules that upregulate expression of one or more of the enzymes of interest e.g. compounds that bind to transcriptional regulatory proteins and modify the binding of the transcriptional regulatory proteins to the regulatory sequences controlling expression of the enzymes of interest. Suitable compounds can be identified, for example, by screening compound libraries and testing for increases in enzyme activity as discussed above.

The streptococcus cells of the invention, and for use in the methods of the invention, are preferably Lancefield group A or group C streptococci, such as Streptococcus equi (for example Streptococcus equi subsp. zooepidemicus or Streptococcus equi subsp. equi). These bacteria naturally produce HA as an extracellular capsule.

### Methods and Cells for Increasing Substrate Levels

The present invention is also based on the unexpected finding that enhanced levels in streptococci of particular substrates involved in the biosynthesis of HA leads to an increase in the molecular weight of the HA produced. One such particular substrate is UDP-N-acetylglucosamine. It has been further determined that enhanced levels of particular substrates such as glucosamine, N-acetylglucosamine and UDP-N-acetylglucosamine, and accordingly an increase in the molecular weight of the HA produced, may be achieved through a variety of methods. These methods include, but are not limited to, provision of additional amounts of the particular substrates or substrate precursors. This may be achieved, for example, by increasing endogenous production of the particular substrates or substrate precursors, or by exogenously increasing bioavailability of the particular substrates or substrate precursors. Other methods for enhancing levels of particular substrates such as glucosamine, N-acetylglucosamine and UDP-N-acetylglucosamine, and accordingly increasing the molecular weight of the HA produced, include, but are not limited to, downregulating or abrogating the activity or amount of enzymes that recruit these substrates or substrate precursors into different biosynthetic pathways, such as UDP-N-acetylglucosamine 1-carboxyvinyltransferase (UDP-NAG-CVT).

In one embodiment, the present invention encompasses methods for producing HA by providing substrate precursors for UDP-NAG. These precursors may include glucosamine, N-acetylgiucosamine and UDP-N-acetylglucosamine. Additionally, such methods further encompass providing metabolites including glutamine, acetyl-CoA and UTP.

Methods for increasing endogenous production of the particular substrates or substrate precursors, such as glucosamine, N-acetylglucosamine and UDP-N-acetylglucosamine, include transforming, transfecting or transducing HA-producing streptococcal cells with an expression vector encoding an enzyme producing said substrate or a precursor thereof. Introduction of the expression vector may be achieved by electroporation, followed by subsequent selection of transformed cells on selective media. Heterologous nucleic acid sequences thereby introduced into the cells may be maintained extrachromosomally or may be introduced into the host cell genome by homologous recombination. Methods for such bacterial cell transformation are well known to those of skill in the art. Guidance may be obtained, for example, from standard texts such as Sambrook et al., Molecular Cloning: A Laboratory Manual, Cold Spring Harbor, N.Y., 1989 and Ausubel et al., Current Protocols in Molecular Biology, Greene Publ. Assoc. and Wiley-Intersciences, 1992.

The present invention therefore provides methods for producing hyaluronic acid which methods comprise growing Streptococcus cells in a culture medium, which cells express the enzymes required for hyaluronic acid synthesis, wherein the cells have been engineered or treated to increase the amount in the cells of one or more substrates selected from glucosamine, N-acetylglucosamine and UDP-N-acetylglucosamine or a precursor thereof; and optionally recovering the hyaluronic acid produced by the cells. The present invention also provides methods for producing hyaluronic acid which comprise recovering hyaluronic acid from Streptococcus cells that express the enzymes required for hyaluronic acid synthesis, wherein the cells have been engineered or treated to increase the amount in the cells of one or more substrates selected from glucosamine, N-acetylglucosamine and UDP-N-acetylglucosamine or a precursor thereof. In preferred embodiments, the substrate is UDP-N-acetylglucosamine.

Methods for increasing bioavailability of the particular substrates or substrate precursors, such as glucosamine, N-acetylglucosamine and UDP-N-acetylglucosamine, include culturing HA-producing streptococcal cells with the substrates or substrate precursors.

Accordingly, the present invention provides methods for producing hyaluronic acid which methods comprise growing Streptococcus cells in a culture medium, which cells express the enzymes required for hyaluronic acid synthesis, and providing one or more substrates selected from glucosamine, N-acetylglucosamine and UDP-N-acetylglucosamine or a precursor thereof; and optionally recovering the hyaluronic acid produced by the cells. The present invention also provides methods for producing hyaluronic acid which comprises recovering hyaluronic acid from Streptococcus cells that express the enzymes required for hyaluronic acid synthesis, wherein one or more substrates selected from glucosamine, N-acetylglucosamine and UDP-N-acetylglucosamine or a precursor thereof has been provided. In preferred embodiments, the substrate is glucosamine.

The present invention hence provides streptococcal cells which comprise the enzymes for synthesis of hyaluronic acid, which cells have been genetically modified to overexpress an enzyme producing one or more substrates selected from glucosamine, N-acetylglucosamine and UDP-N-acetylglucosamine or a precursor thereof. In some embodiments, the overexpression may be achieved by transforming, transfecting or transducing HA-producing streptococcal cells with an expression vector encoding the substrate or a precursor thereof or an enzyme producing said substrate or a precursor thereof. Introduction of the expression vector may be achieved by electroporation, followed by subsequent selection of transformed cells on selective media. Heterologous nucleic acid sequences thereby introduced into the cells may be maintained extrachromosomally or may be introduced into the host cell genome by homologous recombination. In one preferred embodiment, the cells overexpress UDP-N-acetylglucosamine.

Additional methods for maximising the bioavailability of the particular substrates or substrate precursors for use in HA production include providing an alternative substrate with competitive affinity for an enzyme that recruits the substrate into an alternative biosynthesis. For example, provision of a substrate alternative to UDP-N-acetylglucosamine that has competitive affinity for UDP-NAG-CVT will result in recruitment of that substrate by UDP-NAG-CVT for use in peptidoglycan biosynthesis, to the exclusion of UDP-N-acetylglucosamine, thereby allowing for enhanced levels of UDP-N-acetylglucosamine available for HA production.

### Methods and Cells for Decreasing Enzyme Expression or Activity

Methods for downregulating or abrogating the activity or amount of an enzyme in a cell, such as UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase), include disrupting the gene encoding the enzyme such that transcription of the gene is decreased or abrogated, for example, by “knocking out” the gene through insertional or deletional disruption, or through some other form of directed or random mutagenesis that targets either the gene or cofactor involved in transcription of the gene. In this regard, it is significant to note that UDP-NAG-CVT typically exists in HA-producing streptococcal cells in two isoforms, each of which originate from separate genes. Accordingly, it has been determined that one gene encoding UDP-NAG-CVT may be downregulated or abrogated without compromising the viability of the streptococcal cells. Other methods for downregulating or abrogating the activity or amount of an enzyme in a cell include disrupting translation of the mRNA transcribed from the gene, for example, through the use of antisense mRNA or interfering RNA, such siRNA. Further methods for downregulating or abrogating the activity or amount of an enzyme in a cell include targeting the enzyme with an antagonist such a small molecule or an antibody. Methods for such downregulation or abrogation are well known to those of skill in the art, and guidance may be obtained from standard texts such as those disclosed elsewhere herein.

The present invention thus provides methods for producing hyaluronic acid which methods comprise growing Streptococcus cells in a culture medium, which cells express the enzymes required for hyaluronic acid synthesis, wherein the activity or amount in the cells of one or more enzymes selected from UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase) has been decreased or abrogated; and optionally recovering the hyaluronic acid produced by the cells. The present invention also provides methods for producing hyaluronic acid which comprise recovering hyaluronic acid from Streptococcus cells that express the enzymes required for hyaluronic acid synthesis, wherein the activity or amount in the cells of one or more enzymes selected from UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase) has been decreased or abrogated. The present invention further provides methods for producing hyaluronic acid which methods comprise growing Streptococcus cells in a culture medium, which cells express the enzymes required for hyaluronic acid synthesis, wherein the cells have been engineered or treated to decrease or abrogate the activity or amount in the cells of one or more enzymes selected from UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase); and optionally recovering the hyaluronic acid produced by the cells. The present invention moreover provides methods for producing hyaluronic acid which comprise recovering hyaluronic acid from Streptococcus cells that express the enzymes required for hyaluronic acid synthesis, wherein the cells have been engineered or treated to decrease or abrogate the activity or amount in the cells of one or more enzymes selected from UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase).

Decreased or abrogated activity or amount of an enzyme may be measured relative to an equivalent wild-type strain which has not been genetically modified and which is grown under standard conditions (for example, 37° C. in rich media (M17G) or in chemically defined media (CDM) supplemented with 2% w/v D-glucose). For example, in the case of mucoid Group C Streptococcus equi subsp. zooepidemicus, a suitable control strain is ATCC 35246.

The streptococcus cells of the invention, and for use in the methods of the invention are preferably Lancefield group A or group C streptococci, such as Streptococcus equi (for example Streptococcus equi subsp. zooepidemicus or Streptococcus equi subsp. equi). These bacteria naturally produce HA as an extracellular capsule.

The present invention further provides streptococcal cells which comprise the enzymes for synthesis of hyaluronic acid, which cells have been genetically modified to underexpress or not express or express with downregulated activity one or more enzymes selected from UDP-N-acetylglucosamine 1-carboxyvinyltransferase (UDP-NAG-CVT) (murA) or MurG transferase (murG) (undecaprenyldiphospho-muramoylpentapeptide beta-N-acetylglucosaminyltransferase). In some embodiments, the cells comprise one or more mutations in genomic regulatory sequences encoding the one or more enzymes, which mutations result in downregulation or abrogation of expression of the one or more enzymes, relative to a wild type cells. In other embodiments, the cells may comprise one or more mutations in the coding sequences of the one or more enzymes, which mutations result in downregulation or abrogation of expression of the one or more enzymes, relative to a wild type cells. In one preferred embodiment, the enzyme is UDP-NAG-CVT. In another preferred embodiment, the cells may comprise more than one gene encoding UDP-NAG-CVT, and accordingly one gene encoding UDP-NAG-CVT is downregulated or abrogated without compromising the viability of the cells. Such downregulation or abrogation may be achieved by any of the methods described herein. In other embodiments, downregulating or abrogating the activity or amount of an enzyme in a cell is achieved by disrupting translation of the mRNA transcribed from the gene encoding the enzyme, for example, through the use of antibodies directed to the enzyme, or antisense mRNA or interfering RNA, such siRNA. Such antibodies or RNA may be introduced into the cells in an expression vector through methods known to those of skill in the art. In one embodiment, cells have wild type levels and activity of HA synthase (HasA).

The activity of the enzymes of interest can also be downregulated using chemical treatments, e.g. molecules that downregulate expression of one or more of the enzymes of interest e.g. compounds that bind to transcriptional regulatory proteins and modify the binding of the transcriptional regulatory proteins to the regulatory sequences controlling expression of the enzymes of interest. Suitable compounds can be identified, for example, by screening compound libraries and testing for decreases in enzyme activity.

### Cell Culture and Production of Hyaluronic Acid

The present invention provides hyaluronic acid obtained or obtainable by the methods of the invention. HA is produced according to the methods of the invention by culturing suitable streptococci, such as are described above, under suitable conditions. For example, continuous fermentation or a batch fed process may be used. Examples of conditions that can be used to produce HA are described in WO92/08777, which describes a continuous fermentation process with a pH of from 6.0 to 7.0 and dissolved oxygen at less than 1% saturation, and the entire contents of which is incorporated herein by reference. U.S. Pat. No. 6,537,795, the entire contents of which is also incorporated herein by reference, describes a batch fed process. A chemically defined media suitable for the culture of cells is described herein in the examples. Cells are typically cultured at a temperature in a range of from about 35° C. to about 40° C., and more preferably at about 37° C.

Once a desired level of HA production has been achieved in a batch, or at a suitable interval during continuous culture, HA can then be recovered from the cells. A number of methods for purifying HA from bacteria are known in the art. The HA is typically subject to one or more purification steps, particularly where medical grade HA is being produced. The following description, based on U.S. Pat. No. 4,782,046, is by way of example:

Typically the biomass is killed with a suitable agent such as formaldehyde and the HA extracted with an anionic surfactant, such as sodium lauryl sulfate (SLS) or sodium dodecyl sulphate (SDS), or an equivalent anionic detergent, to release the HA from the cells.

The resulting mixture may then simply be filtered, for example through a 0.45 μm mixed cellulose esters filter. An alternative is to treat the mixture with a non-ionic detergent, such as hexadecyltrimethylammonium bromide, or equivalent non-ionic detergent, to precipitate HA and the anionic detergent. The resulting precipitate can be collected via centrifugation or sieve filtration. This precipitate is then solubilised in CaCl₂. The resulting suspension is centrifuged or sieve filtered to remove the precipitate which contains cellular contaminants and both detergents.

The filtrate/supernatant from either method is then extracted with a suitable alcohol (95% EtOH or 99% isopropanol preferred). A gelatinous precipitate forms which is collected via centrifugation or sieve filtration. The pellet is typically washed, for example with an ethanol/saline solution.

Additional purification steps, as described in U.S. Pat. No. 4,782,046, that may be used are as follows: the precipitate is solubilised overnight at 4° C. to 10° C. in deionised, distilled water. The suspension is centrifuged or sieve filtered to remove the precipitate. 1% w/v NaCl is added to the supernatant and dissolved. Then, an appropriate alcohol is added to reprecipitate the HA. Such precipitate is allowed to settle after which it is collected via centrifugation or sieve filtration.

The solubilisation of the HA in water followed by 1.0% NaCl addition and alcohol precipitation may be repeated in increasingly smaller volumes ( 1/20- 1/100 original volume) until the HA-water solution is clear. This may require at least four additional alcohol precipitation steps.

The resulting HA may be sterilised using, for example, 0.1% betapropiolactone (4° C. to 10° C. at 24-48 hours)—the betapropiolactone subsequently being hydrolysed by heating at 37° C.

Other sterilisation methods include filtration using, for example, a suitable protein-binding filter, such as a mixed cellulose esters filter, typically with a pore size of about 0.45 μm.

The resulting bacterial HA of the invention preferably has a MW of more than 3 MDa, preferably more than 3.5 MDa (without being subject to crosslinking).

### Compositions and Methods of Treatment

The HA of the present invention can be used in a variety of applications, such as in cosmetic and reconstructive surgery; in skin anti-ageing, anti-wrinkle products; for replacing biological fluids including synovial fluid (e.g. as an injectable formulation for treating osteoarthritis); for the topical treatment of burns and ulcers; as a surgical aid in cataract extraction, IOL implantation, corneal transplantation, glaucoma filtration, and retinal attachment surgery (e.g. in the form of eye drops or a gel); for adhesion management in surgery, e.g. cardiac surgery, hernia repair, nasal/sinus repair, arthroscopic surgery and spinal surgery; and the like. HA may also be used in speciality foods.

Accordingly the present invention further comprises cosmetic compositions comprising HA obtained or obtainable by the methods of the invention, together with a cosmetically acceptable carrier, excipient or diluent, as well as pharmaceutical compositions comprising HA obtained or obtainable by the methods of the invention, together with a pharmaceutically acceptable carrier, excipient or diluent. Furthermore, the present invention provides food product or food additives comprising the hyaluronic acid of the present invention.

Compositions of the present invention may be administered therapeutically or cosmetically. In a therapeutic application, compositions are administered to a subject already suffering from a condition, in an amount sufficient to cure or at least partially arrest the condition and any complications. The quantity of the composition should be sufficient to effectively treat the patient. Compositions may be prepared according to methods which are known to those of ordinary skill in the art and accordingly may include a cosmetically or pharmaceutically acceptable carrier, excipient or diluent. Methods for preparing administrable compositions are apparent to those skilled in the art, and are described in more detail in, for example, Remington's Pharmaceutical Science, 15th ed., Mack Publishing Company, Easton, Pa., incorporated by reference herein.

Compositions of the present invention may also include topical formulations and/or other therapeutic ingredients. Formulations suitable for topical administration include liquid or semi-liquid preparations suitable for penetration through the skin to the site of where treatment is required, such as liniments, lotions, creams, ointments or pastes, and drops suitable for administration to the eye, ear or nose.

Drops according to the present invention may comprise sterile aqueous or oily solutions or suspensions. These may be prepared by dissolving hyaluronic acid in an aqueous solution of a bactericidal and/or fungicidal agent and/or any other suitable preservative, and optionally including a surface active agent. The resulting solution may then be clarified by filtration, transferred to a suitable container and sterilised. Sterilisation may be achieved by autoclaving or maintaining at 90° C.-100° C. for half an hour, or by filtration, followed by transfer to a container using a sterile technique. Examples of bactericidal and fungicidal agents suitable for inclusion in the drops are phenylmercuric nitrate or acetate (0.002%), benzalkonium chloride (0.01%) and chiorhexidine acetate (0.01%). Suitable solvents for the preparation of an oily solution include glycerol, diluted alcohol and propylene glycol.

Lotions according to the present invention include those suitable for application to the skin. Lotions or liniments for application to the skin may also include an agent to hasten drying and to cool the skin, such as an alcohol or acetone, and/or a moisturiser such as glycerol, or oil such as castor oil or arachis oil.

Creams, ointments or pastes according to the present invention are semi-solid formulations of hyaluronic acid for external application. They may be made by mixing hyaluronic acid in finely-divided or powdered form, alone or in solution or suspension in an aqueous or non-aqueous fluid, with a greasy or non-greasy basis. The base may comprise hydrocarbons such as hard, soft or liquid paraffin, glycerol, beeswax, a metallic soap, a mucilage, an oil of natural origin such as almond, corn, arachis, castor or olive oil, wool fat or its derivatives, or a fatty acid such as stearic or oleic acid together with an alcohol such as propylene glycol or macrogols.

The composition may incorporate any suitable surfactant such as an anionic, cationic or non-ionic surfactant such as sorbitan esters or polyoxyethylene derivatives thereof. Suspending agents such as natural gums, cellulose derivatives or inorganic materials such as silicaceous silicas, and other ingredients such as lanolin, may also be included.

The compositions may also be administered in the form of liposomes. Liposomes may be derived from phospholipids or other lipid substances, and may be formed by mono- or multi-lamellar hydrated liquid crystals dispersed in an aqueous medium. Any non-toxic, physiologically acceptable and metabolisable lipid capable of forming liposomes may be used. The compositions in liposome form may contain stabilisers, preservatives and excipients. Preferred lipids include phospholipids and phosphatidyl cholines (lecithins), both natural and synthetic. Methods for producing liposomes are known in the art, and in this regard specific reference is made to: Prescott, Ed., Methods in Cell Biology, Volume XIV, Academic Press, New York, N.Y. (1976), p. 33 et seq., the contents of which are incorporated herein by reference.

### Dosages

The therapeutically or cosmetically effective dose level for any particular patient will depend upon a variety of factors including the condition being treated and the severity of the condition, the activity of the compound or agent employed, the composition employed, the age, body weight, general health, sex and diet of the patient, the time of administration, the route of administration, the rate of sequestration of the hyaluronic acid, the duration of the treatment, and any drugs used in combination or coincidental with the treatment, together with other related factors well known in the art. One skilled in the art would therefore be able, by routine experimentation, to determine an effective, non-toxic amount of hyaluronic acid which would be required to treat applicable conditions.

Typically, in therapeutic or cosmetic applications, the treatment would be for the duration of the disease state.

Further, it will be apparent to one of ordinary skill in the art that the optimal quantity and spacing of individual dosages of the composition will be determined by the nature and extent of the condition being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

### Routes of Administration

The compositions of the present invention can be administered by standard routes well known to those of skill in the art. The compositions can also be injected directly into synovial joints or a site of inflammation.

### Carriers, Excipients and Diluents

Carriers, excipients and diluents must be “acceptable” in terms of being compatible with the other ingredients of the composition, and not deleterious to the recipient thereof. Such carriers, excipients and diluents may be used for enhancing the integrity and half-life of the compositions of the present invention. These may also be used to enhance or protect the biological activities of the compositions of the present invention.

Examples of pharmaceutically and/or cosmetically acceptable carriers or diluents are demineralised or distilled water; saline solution; vegetable based oils such as peanut oil, safflower oil, olive oil, cottonseed oil, maize oil, sesame oils, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic acceptable diluents or carriers can include Ringer's solution, isotonic saline, phosphate buffered saline, ethanol and 1,2 propylene glycol.

The present invention will now be further described with reference to the following examples, which are illustrative only and non-limiting.

### EXAMPLES

### Example 1

### Materials and Methods

### 1.1 Bacterial Strain

The mucoid Group C Streptococcus equi subsp. zooepidemicus strain ATCC 35246 (S. zooepidemicus) was obtained from the American Type Culture Collection (PO Box 1549, Manassas, Va. 20108, United States of America).

### 1.2 Construction of Recombinant Strains

The 6 genes, namely hasA, hasB, hasC, glmU, pgi, and glmS were amplified from S. zooepidemicus genomic DNA using the primers listed in Table 1. Oligonucleotide primers were designed based on data available from the partial sequence of the Streptococcus equi subspecies zooepidemicus (S. zooepidemicus) has operon available on NCBI (ncbi.nlm.nih.gov: Accession number AF347022) and Sanger Institute S. zooepidemicus Blast Server. Primer GuaB forward and reverse amplify a housekeeping gene of S. zooepidemicus and was used as a polymerase chain reaction (PCR) positive control for S. zooepidemicus. The PCR product sizes were confirmed an agarose gel and the bands extracted using QIAquick Gel Extraction kit (Qiagen). The purified PCR products were double digested with the desired restriction enzymes (see Table 1) and ligated into the nisin inducible plasmid pNZ8148 (Kuipers, O. P., et al. (1998). “Quorum sensing-controlled gene expression in lactic acid bacteria.” J. Biotech. 64(1): 15-21). The ligation mix was used to transform electrocompetent Lactococcus lactis MG1363 and transformants were identified after overnight incubation on M17G agar plates containing 5 μg Cm.ml⁻¹. Colonies were cultured overnight and recombinant plasmids were purified from the pellet using QIAprep Spin Miniprep kit (Qiagen). Insertion site and sequence were confirmed by DNA sequencing. The plasmids were used to transform electrocompetent S. zooepidemicus cells and recombinant strains isolated after overnight culture on M17G agar plates containing 2.5 μg.ml⁻¹ of Cm. The recombinant strains were routinely maintained on sheep blood agar plates containing 2.5 μg.ml⁻¹ Cm.

TABLE 1
Oligonucleotide primers used. Endonuclease
restriction sites are underlined.

| Primer | Sequence (5′-3′) | 5′ site |
|---|---|---|
| HasAF | AGTCCATGGAATACAAAGCGCAAGAAAGGAAC | NcoI |
| | (SEQ ID NO: 1) | |
| HasAR | ATCGCATGCCTCCCTTGTCAGAACCTAGG | SphI |
| | (SEQ ID NO: 2) | |
| HasBF | GTCCATGGAAGAAATGAAAATTTCTGTAGCAGG | NcoI |
| | (SEQ ID NO: 3) | |
| HasBR | ATCGCATGCCTAGTCTCTTCCAAAGACATCT | SphI |
| | (SEQ ID NO: 4) | |
| HasCF | GTCCATGGAAGAACTCATGACAAAGGTCAGAAA | NcoI |
| | AG | |
| | (SEQ ID NO: 5) | |
| HasCR | ATCGCATGCGCTCTGCAATAGCTAAGCCA | SphI |
| | (SEQ ID NO: 6) | |
| GlmUF | GTCCATGGAAAGGAATCAAAACATGAAAAACTA | NcoI |
| | CG | |
| | (SEQ ID NO: 7) | |
| GlmUR | ATCTCTAGAACTATAGCTTACTGGGGCTG | XbaI |
| | (SEQ ID NO: 8) | |
| PgiF | GTCCATGGAAGGGAGTAAAATAATGTCACATAT | NcoI |
| | TACA | |
| | (SEQ ID NO: 9) | |
| PgiR | ATCGCATGCTTACAAGCGTGCGTTGA | SphI |
| | (SEQ ID NO: 10) | |
| GlmSF | ACTCCATGGACGGTGTTAAGTTATGTGTG | NcoI |
| | (SEQ ID NO: 11) | |
| GlmSR | AGCTCTAGATGGCAGGCAACTATTACTCAA | XbaI |
| | (SEQ ID NO: 12) | |
| PgiGlmUF | CATCTAGACGAGGAATCAAAACATGAAAAACTA | XbaI |
| | CG | |
| | (SEQ ID NO: 13) | |
| PgiGlmUR | CAAAGCTTTATAGCTTACTGGGGCTGATCCGGG | HindIII |
| | TGATG | |
| | (SEQ ID NO: 14) | |
| GuaBF | GTTGATGTGGTTAAGGTTGGTATCGG | — |
| | (SEQ ID NO: 15) | |
| GuaBR | AGCCTTGGAAGTAACGGTCGCTTG | — |
| | (SEQ ID NO: 16) | |

### 1.3 Growth Medium and Cultivation Conditions

A single colony was selected from the blood agar plate and inoculated overnight in a chemically defined medium (CDM; Table 2). For pNZ-strains, 2.5 μg.ml⁻¹ of Cland 20 ng.ml⁻¹ of nisin were added to the medium. Growth was monitored at 530 nm with a spectrophotometer.

When an OD₅₃₀ of around 1 was reached, the culture was inoculated to an OD₅₃₀ of 0.05 into a 2 L bioreactor (Applikon). The bioreactor was operated at a working volume of 1.4 L and the temperature maintained at 37° C. The reactor was agitated at 300 rpm and anaerobic conditions maintained by nitrogen sparging during fermentation. pH was controlled at 6.7 by the addition of 5M NaOH and 5 M HCl.

Aerobic culture was also conducted as mentioned above, except with a working volume of 1 L instead of 1.4 L to avoid foam entering the condenser. Aerobic conditions were maintained by constant bottom air sparging at a flow rate of 0.4 L/min during the entire fermentation.

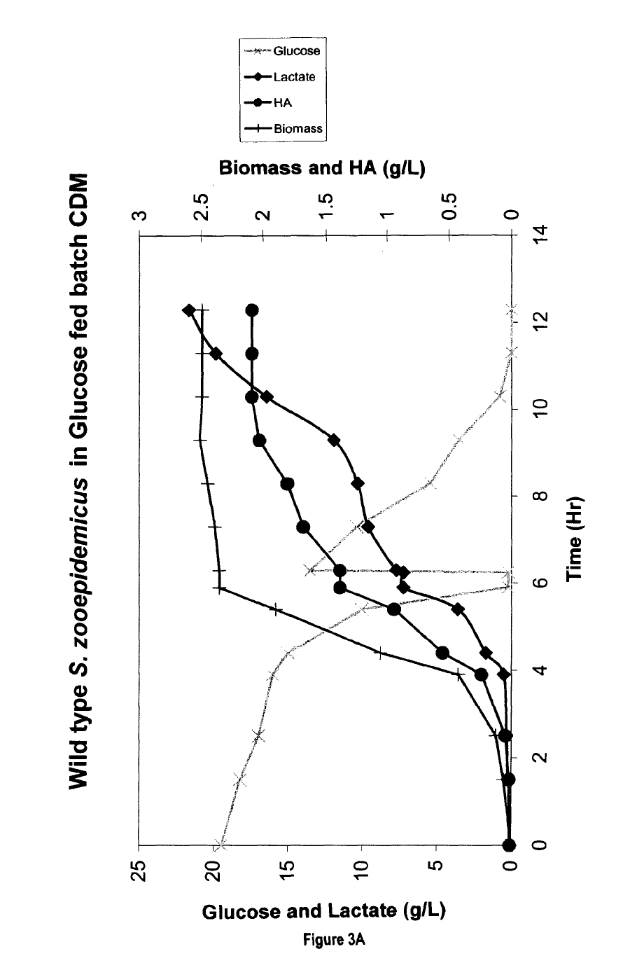

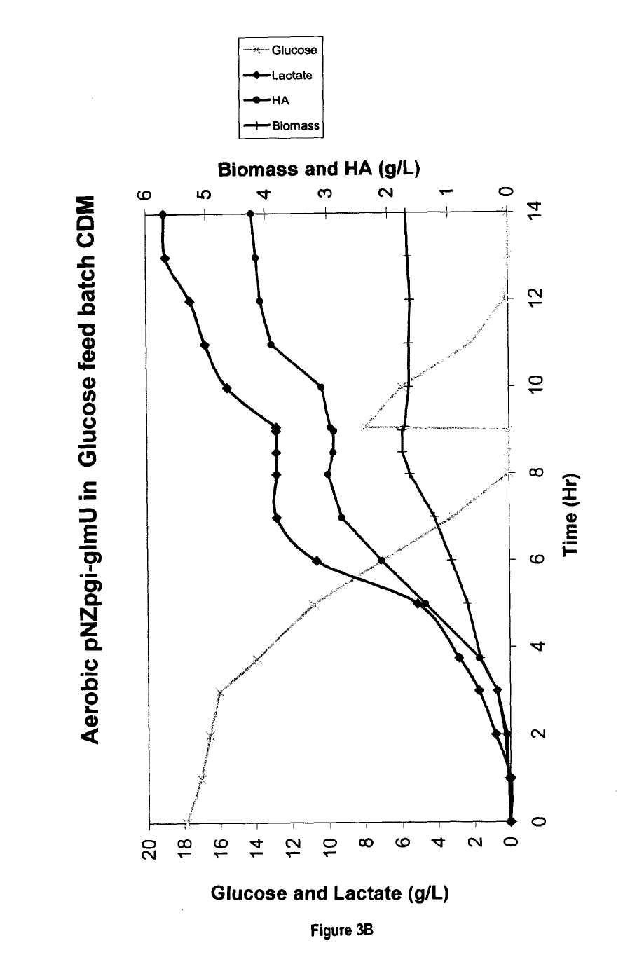

For batch/fed-batch fermentation, the initial batch phase was performed as described above. When the cultures reached stationary phase due to glucose depletion, they were grown for at least an additional 30 mins to ensure complete depletion of an essential amino acid (e.g. arginine through the arginine deiminase pathway). After one hour of stationary phase, additional glucose was added to the cultures, as shown in FIGS. 3A and 3B. With this strategy, as one of the essential amino acids was depleted, biomass could not be synthesized and stationary phase HA production was achieved.

As shown in Table 2, the chemically defined medium (CDM) was modified from Van de Rijn, I. et al. (1980). “Growth characteristics of group A streptococci in a new chemically defined medium.” Infect. Immun. 27(2): 444-448. All chemicals were purchased from Sigma Aldrich.

TABLE 2
Chemically defined medium:

| | | Concentration |
|---|---|---|
| | Component | (mg/L) |
| 1. | FeSO₄•7H₂O | 10 |
| | Fe(NO₃)₂•9H₂O | 1 |
| | K₂HPO₄ | 200 |
| | KH₂PO₄ | 1000 |
| | MgSO₄•7H₂O | 1000 |
| | MnSO₄ | 10 |
| 2. | Alanine | 200 |
| | Arginine | 200 |
| | Aspartic acid | 200 |
| | Asparagine | 200 |
| | Cystine | 100 |
| | Glutamic acid | 200 |
| | Glutamine | 5600 |
| | Glycine | 200 |
| | Histidine | 200 |
| | Isoleucine | 200 |
| | Leucine | 200 |
| | Lysine | 200 |
| | Methionine | 200 |
| | Phenylalanine | 200 |
| | Proline | 200 |
| | Hydroxy-L-proline | 200 |
| | Serine | 200 |
| | Theonine | 400 |
| | Tryptophan | 200 |
| | Tyrosine | 200 |
| | Valine | 200 |
| 3 | Glucose | 20000 |
| 4. | Uridine | 50 |
| | Adenine | 40 |
| | Guanine | 40 |
| | Uracil | 40 |
| 5. | CaCl₂•6 H₂O | 10 |
| | NaC₂H₃O₂•3H₂O | 4500 |
| | Cysteine | 500 |
| | NaHCO₃ | 2500 |
| | NaH₂PO₄•H₂0 | 3195 |
| | Na₂HPO₄ | 7350 |
| 6. | p-Aminobenzoic acid | 0.2 |
| | Biotin | 0.2 |
| | Folic acid | 0.8 |
| | Niacinamide | 1 |
| | B-NAD | 2.5 |
| | Pantothenate Ca salt | 2 |
| | Pyridoxal | 1 |
| | Hydrochloride | |
| | Pyridoxamine | 1 |
| | hydrochloride | |
| | Riboflavin | 2 |
| | Thiamine | 1 |
| | hydrochloride | |
| | Vitamin B12 | 0.1 |
| | Inositol | 2 |

### 1.4 Measurement of Biomass and Fermentation Products

Samples were collected hourly and the optical density measured with a spectrophotometer at a wavelength of 530 nm and converted to biomass using the equation: Biomass (g/L)=OD530* 0.26±0.01 (Goh, L.-T. (1998). Fermentation studies of Hyaluronic acid production by Streptococcus zooepidemicus. Department of Chemical Engineering. Brisbane Australia). The remaining sample was mixed with an equal volume of SDS to break the HA capsule and filtered through a syringe filter (0.45 μm) for cell removal.

Lactic acid, acetate, formate, glucose and ethanol were measured by HPLC using a BioRad HPX-87 H acid column with 1M H₂SO₄ as eluent and a flow rate of 1 mL per minute. Samples with glucose concentrations below 40 ppm were analysed using a YSI 2700 Select Biochemistry Glucose Analyser (Yellow Springs Inc.).

The concentration of the HA sample was measured using a HA turbidimetric quantification assay (Di Ferrante, N. (1956). “Turbidimetric measurement of acid mucopoly-saccharides and hyaluronidase activity.” J. Bio. Chem. 220: 303-306). Briefly, 200 μL of the sample was mixed with 200 μL of 0.1 M potassium acetate (pH=5.6) and 400 μL of 2.5% w/v cetyl-trimethyl-ammonium-bromide (CTAS) in 0.5 M NaOH. After 20 minutes of incubation, the OD600 was determined and the HA concentration determined from a calibration curve.

### 1.5 Measurement of UDP Sugars

Five ml of cell suspension was pelleted by centrifugation (50,000×g, 2 min, 37° C.) and extracted with boiling ethanol. Extracts were processed via solid phase extraction using 500 mg SAX resin columns (6 ml reservoir, Isolute, International Sorbent Technology) as described elsewhere (Jensen, N. B. S., Jokumsen, K. V., Villadsen, J., Determination of the phosphorylated sugars of the Embden-Meyerhoff-Parnas pathway in Lactococcus lactis using a fast sampling technique and solid phase extraction. Biotechnol. Bioeng. 1999, 63, 356-362) except that metabolites were eluted from columns using 2 mL of 0.15 M sodium citrate instead of sodium acetate.

Samples were diluted 1:1 (w/w) with water before UDP-sugar analysis by high pressure anion exchange chromatography (HPAEC). 25 μL of diluted samples were injected into an AAA Direct system (Dionex, Sunnyvale, USA) fitted with an AminoTrap guard column (2 mm×50 mm) and a CarboPac PA10 analytical column (2 mm×250 mm) (Dionex, Sunnyvale, USA). Column temperature was maintained at 30° C. and the flow rate was set at 0.25 ml min⁻¹. UDP-sugars were eluted with a sodium acetate gradient in 1 mM NaOH and detected using an ED40 electrochemical detector with a gold electrode (Dionex, Sunnyvale, USA).

### 1.6 Assay of Enzyme Activities

hasA activity was assayed by in vivo synthesis of HA from membrane extract obtained using a protocol based on a previously described method (Tlapak-Simmons, V. L., Baggenstoss, B. A., Kumari, K., Heldermon, C., and Weigel, P. H. (1999). Kinetic Characterization of the Recombinant Hyaluronan Synthases from Streptococcus pyogenes and Streptococcus equisimilis. J Biological Chemistry 274, 4246-4253). Initially, 400 μL of membrane lysate, was mixed with 200 μL of 4 mM UDP-Glucuronic acid dissolved in wash buffer (50 mM KH₂PO₄, 5 mM EDTA, 10% Glycerol, protease inhibitors mixture (GE healthcare), pH 7)) and 400 μL of 4 mM UDP-N-acetyl glucosamine (in wash buffer). Subsequently, 100 μL of HAS buffer (250 mM Na₂HPO₄, 250 mM KH2PO₄, 500 mM NaCl, 1 mM EGTA), 20 μL of 1 M MgCl₂, 20 μL of 20 mM DTT, 10 μL protease inhibitors mixture (GE healthcare) and 50 μL of wash buffer were added to the reactants. The enzymatic reaction was maintained at 37° C. in a water bath for 2 hours and subsequently in a 100° C. water bath for 2 minutes to terminate the reaction (Tlapak-Simmons, et al. (1999) ibid). After cooling to room temperature, 1 mL of 0.1% SDS was added to free the HA attached to the membrane extract and HA was measured by the Turbidimetric assay described above.

Other enzyme activities were assayed using protocols based on previously described methods: HasB (Dougherty, B. and van de Rijn, I. (1993). “Molecular characterization of hasB from an operon required for hyaluronic acid synthesis in group A streptococci. Demonstration of UDP-glucose dehydrogenase activity.” J. Biol. Chem. 268(10): 7118-7124), HasC (Franke, J. and Sussman, M. (1971). “Synthesis of Uridine Diphosphate Glucose Pyrophosphorylase during the Development of Dictyostelium discoideum.” J. Biol. Chem. 246(21): 6381-6388), and Pgi (Bergmeyer, H. U., et al. (1974). Methods of Enzymatic Analysis (Bergmeyer, H. U., ed.). New York, N.Y., Academic Press, Inc.).

GlmU activity was not determined, but expression was confirmed using real-time PCR. RNA was purified from the cell extracts using the RNeasy mini kit (Qiagen), DNase treated and subjected to RT-PCR with the SuperScript One-Step RT PCR kit (Gibco) using primers: GImUF (5′-GTCCATGGAAAGGAATCAAAACATGAAAAACTACG-3′) (SEQ ID NO: 7) and GImUR (5′-ATCTCTAGAACTATAGCTTACTGGGGCTG-3′) (SEQ ID NO: 8). After 24 cycles, the resultant 1396 bp DNA fragment of the glmU gene was quantified on an agarose gel based on band intensity (Scion Image Beta 4.0.3).

### 1.7 Molecular Weight Determination

HA samples were purified from the broth by mixing 15 mL of culture with 15 mL of 0.1% w/v SDS incubated at room temperature for 10 minutes (Chong, B. F. (2002). Improving the cellular economy of Streptococcus zooepidemicus through metabolic engineering. Department of Chemical Engineering. Brisbane, The University of Queensland). Samples were then filtered through a 0.45 μm filter and the filtrates were thawed and mixed with 3 volumes of ethanol and left overnight at 4° C. The precipitates were then centrifuged (9630×g; 4° C.; 20 min) and supernatant removed. The pellet was washed in 15 mL ethanol; saline solution (75% w/v ethanol, 25% w/v 0.15M NaCl) and again centrifuged (17600×g; 4° C.; 20 min). After removal of the supernatant, the pellet was allowed to dry overnight. Finally, the HA pellet was then resuspended in 0.15 M NaCl with gentle rocking and undissolved matter was removed by centrifugation (17600×g; 4° C.; 20 min) and samples were filtered through 0.45 μm filter.

Intrinsic viscosity was measured with a Lauda Processor viscosity measuring system using an Ubbelohde Dilution Capillary (0.63 mm diameter, 5700 mm³ volume). All measurements were performed at 37° C. and 0.15M sodium chloride was used as diluting solvent. The intrinsic viscosity was used to determine the average molecular weight using the Mark-Houwink-Sakurada equation: [η]=0.0292× M̅w^{0.7848}, fitted using standards of known molecular weight processed as outlined above.

### 1.8 Proteomics

200 mL of cells in exponential growth (OD₅₃₀=2-4) were harvested into a Schott bottle containing 20 mg of hyaluronidase and incubated at 37° C. for 10 minutes. Cells were pelleted at 20,000×g (20 min, 4° C.; Avanti J26 XPI, Beckman Coulter) and resuspended in 30 ml of lysis buffer (30 mM Tris, 7M urea, 2M thiourea, 4% CHAPS and protease inhibitors cocktails). Cells were lysed on a bead beater with 1.44 g of 100 μm glass beads. Samples were cleaned up using a 2-D clean up kit and protein concentration determined using a 2-D Quant kit according to the manufacturer's protocol (GE Healthcare). 50 μg of proteins were labelled using CyDYE labelling kit according to the manufacturer's protocol (GE-Healthcare).

Isoelectric focusing was performed using IPG strips (GE-Healthcare, 24 cm). Proteins were separated on a Multiphore I unit (GE, Healthcare) by active rehydration, (30V) for 12 hours prior to isoelectric focusing: 1 h, 500V (Step and hold); 1 h, 1000 V (gradient); 3 h, 8000V (gradient); 12 h, 8000V (Step and hold). After equilibration, IPG strips were transferred to the second dimension SDS-PAGE using polyacrylamide gels on an Ettan Dalt 12 electrophoresis unit (GE Healthcare) with 2 w/gel for 30 minutes and 18 W/gel for 6 h. Gel images were scanned using Typhoon trio 9100 (GE Healthcare) at 100 μm according to the manufacturer's protocol. Proteins were identified using mass spectrometry (LC/MS/MS and MALDI TOF/TOF).

### 1.9 Mass Spectrometry

Protein spots were excised from the gel and in-gel digested with an excess of trypsin (Promega, Trypsin Gold, MS grade) (overnight at 37° C.). Peptides were dried using a SpeediVac (SPD111V, Tthermo Savant) and re-dissolved in 80 μL of 5% formic acid for MS analysis. An Agilent 1100 Binary HPLC system (Agilent), was used to perform reversed phase separation of the samples prior to MS using a Vydac MS C18 300 A, column (150 mm×2 mm) with a particle size of 5 μm (Vydac). Eluate from the RP-HPLC column was directly introduced into the TurbolonSpray source.

Mass spectrometry experiments were performed on a hybrid quadrupole/linear ion trap 4000 QTRAP MS/MS system (Applied Biosystems). The 4000 QTRAP equipped with a TurbolonSpray Source was operated in positive electrospray ionization mode. Analyst 1.4.1 software was used for data analysis. The acquisition protocol used to provide mass spectral data for database searching involved the following procedure: mass profiling of the HPLC eluant using Enhanced Multiple Scan (EMS). The most and next most abundant ions in each of these scans with a charge state of +2 to +3 or with unknown charge were subjected to CID using a rolling collision energy. An Enhanced product ion scan was used to collate fragment ions and present the product ion spectrum for subsequent database searches.

Additionally, some samples were analysed using MALDI-MS using a 4700 Proteomics Analyzer MALDI-TOF/TOF (Applied Biosystems). When necessary, the samples were first desalted using micro C18 ZipTips (Millipore), and peptides eluted directly with 5 mg/mL of CHCA in 60% ACN/0.1% formic acid onto a MALDI target plate. All MS spectra were recorded in positive reflector mode at a laser energy of 4800. All MS/MS data from the TOF-TOF was acquired using the default positive ion, 1 kV collision energy, reflectron mode, MS/MS method at a laser energy of 5500. The TOF-MS spectra were analyzed using the Peak Picker software supplied with the instrument. The 10 most abundant spectral peaks that met the threshold (>20:1 signal:noise) criteria and were not on the exclusion list were included in the acquisition list for the TOF-TOF, MS/MS portion of the experiment. The threshold criteria were set as follows: mass range: 500 to 4000 Da; minimum cluster area: 500; minimum signal-to-noise (S/N): 20; maximum number of MS/MS spectra per spot: 10. A mass filter excluding matrix cluster ions and trypsin autolysis peaks was applied.

Database searching of LC-MS/MS and non-interpreted TOF-MS and TOF-TOF MS/MS data was carried out using the ProteinPilot software (version 2.0.1) and Paragon algorithm (Applied Biosystems)

### Example 2

### Results

### 2.1 Overexpression of Enzymes Enhancing HA Molecular Weight

Seven genetically modified S. equi strains (hasA, hasB, hasC, glmU, pgi, glmS and pgi-glmU) were generated as outlined in Materials and Methods. Overexpression of genes was confirmed using enzyme assays (hasA, hasB, hasC, glmS and pgi) or RT-PCR (glmU). Each strain was fermented in a bioreactor and the molecular weight of HA produced determined using viscometry. Each engineered strain produced HA of a molecular weight greater than that of the wildtype strain (Table 3). The increases, however, were partly attributable to the plasmid; strains carrying the pNZ8148 plasmid with a nisA promoter used for overexpression or a similar plasmid pNZ9530 with a nisRK promoter in which the chloramphenicol marker had been replaced with an erythromycin marker showed increased HA molecular weight compared to wildtype (WT).

Relative to the empty plasmid strains, only the strains carrying genes involved in the UDP-NAG pathway (pgi, glmS and glmU) displayed higher MW. Moreover, another strain engineered to overexpress both pgi and glmU produced the highest molecular weight of all strains. Consistent with this observation, HA MW correlated strongly (0.86) with the levels of UDP-NAG, but not with UDP-GUA levels (0.07).

TABLE 3
UDP sugar levels and % increase in molecular weight
of HA produced by genetically modified S. equi strains
(relative to wild type value of 1.77 MDa)

| Strain | UDP-NAG | UDP-GUA | MW |
|---|---|---|---|
| WT | 0.89 | 0.59 | 100% |
| pNZ8148 | 1.07 | 0.88 | 131% |
| pNZ9530 | | | 122% |
| hasA⁺⁺ | 0.86 | 0.51 | 117% |
| hasB⁺⁺ | 1.08 | 11.05 | 124% |
| hasC⁺⁺ | 0.58 | 7.83 | 110% |
| glmU⁺⁺ | 1.19 | 1.02 | 145% |
| pgi⁺⁺ | 1.40 | 0.97 | 178% |
| glmU⁺⁺pgi⁺⁺ | 1.79 | 1.48 | 203% |
| glmS | 0.96 | 0.87 | 158% |

2.2 Proteomics Analysis of WT, Empty Plasmid (pNZ8148) and pgi⁺⁺ Strains

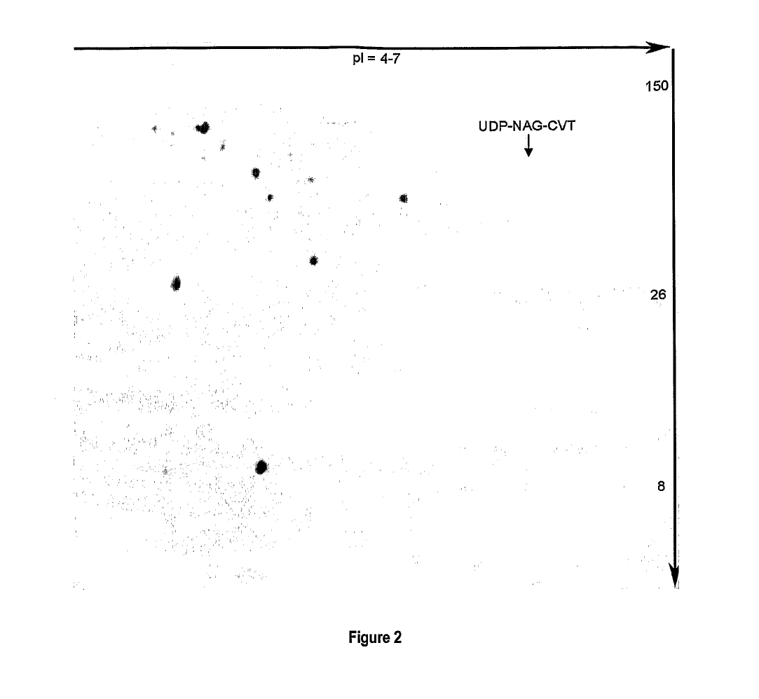

Proteomics was used to identify the mechanism by which the empty plasmid increases UDP-NAG levels and hereby molecular weight. The wild type (WT), empty plasmid (pNZ8148) and pe+strains were compared using DICE proteomics (FIG. 2).

The abundance of ten protein spots was significantly different between the wild type and the empty plasmid (pNZ8148) cultures (Table 4) as per ANOVA testing. Seven of these spots could not be identified by MS due to low abundance in the preparative coomasie gel. Spot 24 was mapped to the two homologues of UDP-N-acetylglucosamine 1-carboxyvinyltransferase (UDP-NAG-CVT) found in the S. zooepidemicus genome. Using LC/MS/MS 5 peptides were mapped to one of the genes and 3 peptides to the other. UDP-NAG-CVT catalyses the first step in peptidoglycan biosynthesis from UDP-NAG and represents the major non-HA associated drain of UDP-NAG. Spot 56 was mapped to UDP-N-acetyl-glucosamine pyrophosphorylase (GlmU). A significant increase in GlmU together with a significant decrease in UDP-NAG-CVT may explain why the empty plasmid strain has higher UDP-NAG concentration and higher MW than the wildtype (WT).

TABLE 4
Significant results of the proteome analysis comparing
wild type versus empty plasmid (pNZ8148).

| | | | | Fold |
|---|---|---|---|---|
| Spot | Protein Description | Gene ID | p value | increase |
| 48 | Unidentified protein | NID | 0.0332 | 4.6 |
| 24 | UDP-N-acetylglucosamine-1-carboxyvinyltransferase | SZ2160 | 0.0185 | −3.4 |
| 56 | UDP-N-acetyl-glucosamine pyrophosphorylase | SZ1872 | 0.0346 | 2.9 |
| 999 | Unidentified protein | NID | 0.0409 | 2.1 |
| 11 | 3-ketoacyl-(acyl-carrier-protein)reductase | SZ0340 | 0.0244 | 2 |
| 228 | Unidentified protein | NID | 0.0014 | 1.9 |
| 505 | Unidentified protein | NID | 0.0453 | −1.8 |
| 219 | Unidentified protein | NID | 0.0284 | 1.8 |
| 201 | Unidentified protein | NID | 0.0310 | 1.7 |
| 152 | Unidentified protein | NID | 0.0294 | 1.7 |

Proteins not identified are marked as NID.

Compared to the empty plasmid strain, the pgi⁺⁺ strain displayed a further 1.8-fold increase in glmU (Table 5). Several proteins were significantly different, however proteins could not be identified by MS. Two unidentified proteins (spot 48 and 201) displayed a similar pattern, i.e., increased abundance in the empty plasmid strain compared to wildtype and a further increase between empty plasmid and pgi⁺⁺ strains. Two spots (95 and 463) that mapped to Pgi showed the expected increase, though a definite conclusion was hampered by the contamination with other proteins in these spots.

TABLE 5
Significant results of the proteome analysis comparing
empty plasmid (pNZ8148) versus pgi strain.

| | | | | Fold |
|---|---|---|---|---|
| Spot | Protein Description | Gene ID | p value | increase |
| 48 | Unidentified protein | NID | 0.0308 | 2.7 |
| 95 | Glucose-6-phosphate isomerase | SZ1874 | 0.0461 | 2.3 |
| | putative S-adenosylmethionine synthase | SZ0660 | | |
| 201 | Unidentified protein | NID | 0.0458 | 1.9 |
| 56 | UDP-N-acetyl-glucosamine pyrophosphorylase | SZ1872 | 0.0123 | 1.8 |
| 463 | Glucose-6-phosphate isomerase | SZ1874 | 0.0396 | 1.4 |
| | Phosphopyruvate hydratase | SZ0823 | | |
| | putative Amidopeptidase C | SZ1725 | | |
| | NADH oxidase | SZ1094 | | |
| 16 | Hypothetical protein | SZ0352 | 0.0284 | 1.3 |

Proteins not identified are marked as NID.

### 2.3 Aerobic Conditions Further Increases Molecular Weight

Two mutant strains carrying pgi and the dual genes pgi and glmU were compared to wild type under aerobic conditions. Aerobic conditions had little effect on HA yield, slightly reduced the growth rate but increased HA MW significantly, as shown in Table 6.

TABLE 6
Percentage increase in molecular weight of HA produced
by aerobic fermentation S. equi strains (MDa)

| | MW | MW | |
|---|---|---|---|
| | anaerobic | Aerobic | % |
| Strain | conditions | conditions | increase |
| WT | 1.77 | 2.27 | 28% |
| pgi⁺⁺ | 3.17 | 3.86 | 21% |
| glmU⁺⁺pgi⁺⁺ | 3.44 | 4.26 | 23% |

### 2.4 Batch-Fed-Batch Fermentation to Achieve a Stationary HA Production of High MW.

In order to further increase HA MW by process optimization, a batch/fed-batch strategy was undertaken. This strategy involved a brief period of glucose starvation between batches so as to effect arginine depletion. As a proof of concept demonstration, wild type streptococci were cultured under anaerobic conditions (FIG. 3A). HPLC analysis showed that arginine was rapidly depleted once glucose was depleted at the end of the batch period. HA production but not cell growth resumed after feeding.

The average molecular weight at the end of the fed-batch fermentation was 2.4 MDa compared to 1.8 MDa under batch conditions. As can be seen in FIG. 3A, 66% of HA was produced under the batch fermentation and 34% under stationary phase, from which it can be inferred that HA produced during the stationary phase had an average MW of 3.6 MDa.

In order to conduct optimal fermentation, the strain carrying the dual genes pgi-glmU was tested under aerobic conditions, as shown in FIG. 3B. Using the fed-batch strategy, 5.0 MDa was obtained, as shown in Table 7.61% of the HA was produced under the batch at an average MW of 4.2 MDa. The remaining 39% was produced in stationary phase, with an average MW of 6.4 MDa.

TABLE 7
Percent increase in molecular weight of HA produced by
fed-batch aerobic fermentation S. equi strains (MDa)

| | Average | % |
|---|---|---|
| Strain | MW | increase |
| WT | 2.4 | 33% |
| Anaerobic | | |
| glmU⁺⁺pgi⁺⁺ | 5.0 | 18% |
| Aerobic | | |

### 2.5 Fermentation on Glucosamine

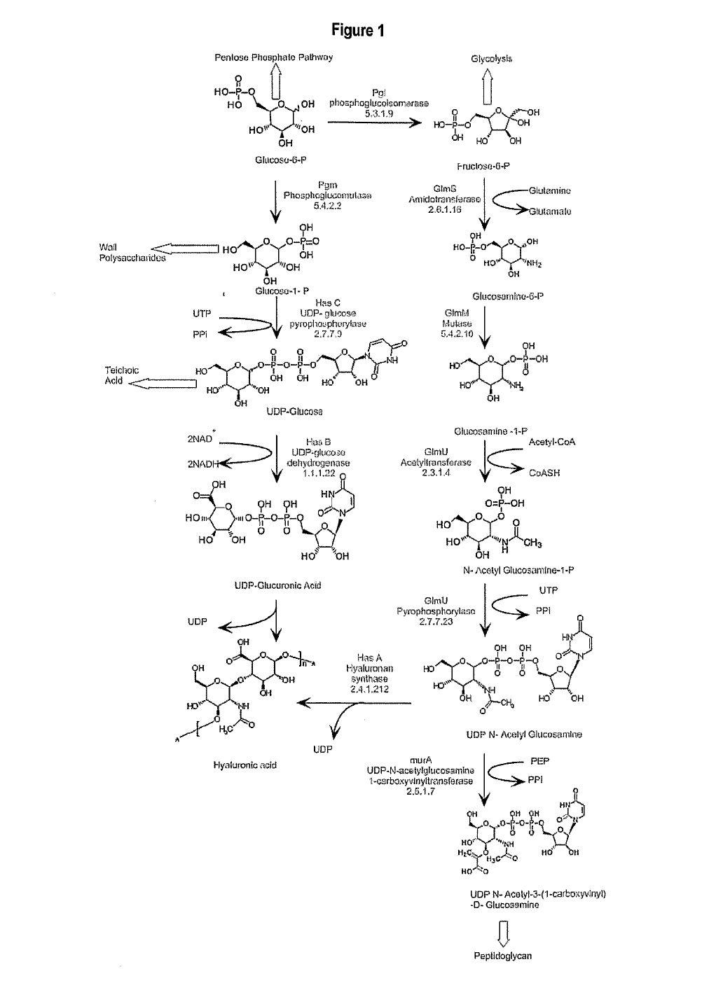

If metabolised, glucosamine is expected to be transported by a phosphotransferease system producing glucosamine-6-phosphate in the process. Glucosamine-6-phosphate is part of the UDP-NAG pathway (FIG. 1), thus feeding glucosamine should increase UDP-NAG levels.

S. zooepidemicus grew well on CDM in which glucose was replaced with glucosamine. Measured UDP-NAG levels were two times greater than seen on glucose based medium. UDP-GUA concentrations, however, were below detection and the MW was only 1.5 MDa. This indicates that while glucosamine can be fed to enhance UDP-NAG levels care must be made to ensure that UDP-GUA is not depleted. For example, the culture may be supplied by a mixture of glucose and glucosamine to balance the supply of the two precursors.

### Example 3

### Conclusion

The inventors have described the design and construction of a number of streptococcal strains that overexpress specific enzymes in the HA biosynthetic pathway, and which are capable of synthesizing significantly higher MW HA compared to wild type strains.

All strains produced HA of higher molecular weight compared to the wildtype, but only strains overexpressing genes in the UDP-NAG pathway produced HA of higher molecular weight than the empty plasmid control. It was observed that molecular weight correlated strongly with UDP-NAG levels, but not with UDP-GUA levels. A higher level of UDP-NAG and hence molecular weight in the empty plasmid control compared to the wildtype strain was attributed to lower competition for UDP-NAG for peptidoglycan biosynthesis; DIGE proteomics identified a significant reduction in the empty plasmid control in the levels of UDP-NAG-CVT, which catalysis the first UDP-NAG utilising step in peptidoglycan biosynthesis.

The various features and embodiments of the present invention, referred to in individual sections above apply, as appropriate, to other sections, mutatis mutandis. Consequently features specified in one section may be combined with features specified in other sections as appropriate.

All publications mentioned in the above-specification are herein incorporated by reference. Various modifications and variations of the described methods and products of the invention will be apparent to those of skill in the art without departing from the spirit and scope of the invention. Although the invention has been described in connection with specific preferred embodiments, it should be understood that that the invention as claimed should not be unduly limited to such specific embodiments. Indeed, in various modifications of the described modes for carrying out the invention which are apparent to those skilled in the relevant fields are intended to be within the scope of the following claims.

1. A method for producing hyaluronic acid, wherein the method comprises growing Streptococcus cells in a culture medium, wherein the cells express the enzymes required for hyaluronic acid synthesis, wherein the activity or amount in the cells of one or more enzymes selected from:
(a) phosphoglucoisomerase;
(b) D-fructose-6-phosphate amidotransferase;
(c) phosphoglucosamine mutase;
(d) glucosamine-1-phosphate acetyl transferase;
(e) N-acetylglucosamine-1-phosphate pyrophosphorylase
(f) glucosamine-6-phosphate acetyl transferase; and
(g) phosphoacetylglucosamine mutase
has been increased, thereby producing hyaluronic acid.

2. The method according to claim 1, further comprising recovering the hyaluronic acid produced by the cells.

3. A method for producing hyaluronic acid, wherein the method comprises recovering hyaluronic acid from Streptococcus cells that express the enzymes required for hyaluronic acid synthesis, wherein the activity or amount in the cells of one or more enzymes selected from:
(a) phosphoglucoisomerase;
(b) D-fructose-6-phosphate amidotransferase;
(c) phosphoglucosamine mutase;
(d) glucosamine-1-phosphate acetyl transferase;
(e) N-acetylglucosamine-1-phosphate pyrophosphorylase
(f) glucosamine-6-phosphate acetyl transferase; and
(g) phosphoacetylglucosamine mutase
has been increased.

4. A method for producing hyaluronic acid, wherein the method comprises growing Streptococcus cells in a culture medium, wherein the cells express the enzymes required for hyaluronic acid synthesis, wherein the cells have been engineered or treated to increase the activity or amount in the cells of one or more enzymes selected from:
(a) phosphoglucoisomerase;
(b) D-fructose-6-phosphate amidotransferase;
(c) phosphoglucosamine mutase;
(d) glucosamine-1-phosphate acetyl transferase;
(e) N-acetylglucosamine-1-phosphate pyrophosphorylase
(f) glucosamine-6-phosphate acetyl transferase; and
(g) phosphoacetylglucosamine mutase
thereby producing hyaluronic acid.

5. The method according to claim 4, further comprising recovering the hyaluronic acid produced by the cells.

6. A method for producing hyaluronic acid, wherein the method comprises recovering hyaluronic acid from Streptococcus cells that express the enzymes required for hyaluronic acid synthesis, wherein the cells have been engineered or treated to increase the activity or amount in the cells of one or more enzymes selected from:
(a) phosphoglucoisomerase;
(b) D-fructose-6-phosphate amidotransferase;
(c) phosphoglucosamine mutase;
(d) glucosamine-1-phosphate acetyl transferase;
(e) N-acetylglucosamine-1-phosphate pyrophosphorylase
(f) glucosamine-6-phosphate acetyl transferase; and
(g) phosphoacetylglucosamine mutase
thereby producing hyaluronic acid.

7. The method according to claim 1, wherein the activity or amount in the cells of the one or more enzymes produces more UDP-N-acetyl glucosamine compared to wild type Streptococcus cells.

8. The method according to claim 1, wherein the hyaluronic acid produced is of a higher average molecular weight compared to wild type Streptococcus cells.

9. A method for producing hyaluronic acid, wherein the method comprises growing Streptococcus cells in a culture medium, wherein the cells express the enzymes required for hyaluronic acid synthesis; and providing one or more substrates selected from:
(a) UDP-N-acetylglucosamine;
(b) N-acetylglucosamine; and
(c) glucosamine
thereby producing hyaluronic acid.

10. The method according to claim 9, further comprising recovering the hyaluronic acid produced by the cells.

11. A method for producing hyaluronic acid, wherein the method comprises recovering hyaluronic acid from Streptococcus cells that express the enzymes required for hyaluronic acid synthesis, wherein one or more substrates selected from:
(a) UDP-N-acetylglucosamine;
(b) N-acetylglucosamine; and
(c) glucosamine
has been provided.

12. The method according to claim 9, further comprising providing one or more metabolites selected from:
(a) glutamine;
(b) acetyl-CoA; and
(c) UTP.

13. A method for producing hyaluronic acid, wherein the method comprises growing Streptococcus cells in a culture medium, wherein the cells express the enzymes required for hyaluronic acid synthesis, wherein the cells have been engineered or treated to increase the amount in the cells of one or more substrates selected from:
(a) UDP-N-acetylglucosamine;
(b) N-acetylglucosamine; and
(c) glucosamine
thereby producing hyaluronic acid.

14. The method according to claim 13, further comprising recovering the hyaluronic acid produced by the cells.

15. A method for producing hyaluronic acid, wherein the method comprises recovering hyaluronic acid from Streptococcus cells that express the enzymes required for hyaluronic acid synthesis, wherein the cells have been engineered or treated to increase the amount in the cells of one or more substrates selected from:
(a) UDP-N-acetylglucosamine;
(b) N-acetylglucosamine; and
(c) glucosamine.

16. The method according to claim 13, wherein the cells have been engineered or treated to increase the amount in the cells of one or more metabolites selected from:
(a) glutamine;
(b) acetyl-CoA; and
(c) UTP.

17. The method according to claim 9, wherein the amount in the cells of UDP-N-acetyl glucosamine is higher compared to wild type Streptococcus cells.

18. The method according to claim 9, wherein the hyaluronic acid produced is of a higher average molecular weight compared to wild type Streptococcus cells.

19. A method for producing hyaluronic acid, wherein the method comprises growing Streptococcus cells in a culture medium, which cells express the enzymes required for hyaluronic acid synthesis, wherein the activity or amount in the cells of one or more enzymes selected from:
(a) UDP-N-acetylglucosamine 1-carboxyvinyltransferase; and
(b) undecaprenyldiphospho-muramoylpentapeptide beta-N-acetylglucosaminyltransferase
has been decreased or abrogated, thereby producing hyaluronic acid.

20. The method according to claim 19, further comprising recovering the hyaluronic acid produced by the cells.

21. A method for producing hyaluronic acid, wherein the method comprises recovering hyaluronic acid from Streptococcus cells that express the enzymes required for hyaluronic acid synthesis, wherein the activity or amount in the cells of one or more enzymes selected from:
(a) UDP-N-acetylglucosamine 1-carboxyvinyltransferase; and
(b) undecaprenyldiphospho-muramoylpentapeptide beta-N-acetylglucosaminyltransferase
has been decreased or abrogated.

22. A method for producing hyaluronic acid, wherein the method comprises growing Streptococcus cells in a culture medium, wherein the cells express the enzymes required for hyaluronic acid synthesis, wherein the cells have been engineered or treated to decrease or abrogate the activity or amount in the cells of one or more enzymes selected from:
(a) UDP-N-acetylglucosamine 1-carboxyvinyltransferase; and
(b) undecaprenyldiphospho-muramoylpentapeptide beta-N-acetylglucosaminyltransferase
thereby producing hyaluronic acid.

23. The method according to claim 22, further comprising recovering the hyaluronic acid produced by the cells.

24. A method for producing hyaluronic acid, wherein the method comprises recovering hyaluronic acid from Streptococcus cells that express the enzymes required for hyaluronic acid synthesis, wherein the cells have been engineered or treated to decrease or abrogate the activity or amount in the cells of one or more enzymes selected from:
(a) UDP-N-acetylglucosamine 1-carboxyvinyltransferase; and
(b) undecaprenyldiphospho-muramoylpentapeptide beta-N-acetylglucosaminyltransferase.

25. The method according to claim 19, wherein at least one copy of a gene encoding UDP-N-acetylglucosamine 1-carboxyvinyltransferase in the cells has been mutated to underexpress or not express or express with downregulated activity UDP-N-acetylglucosamine 1-carboxyvinyltransferase.

26. The method according to claim 19, wherein the activity or amount of the one or more enzymes results in less use of UDP-N-acetyl glucosamine by the one or more enzymes compared to wild type Streptococcus cells.

27. The method according to claim 19, wherein the hyaluronic acid produced is of a higher average molecular weight compared to wild type Streptococcus cells.

28. Hyaluronic acid obtained or obtainable according to the method of claim 1.

29. The hyaluronic acid according to claim 28, having an average molecular weight of at least 3 MDa.

30. The hyaluronic acid according to claim 28, having substantially no crosslinking.

31. A Streptococcus cell comprising the enzymes for synthesis of hyaluronic acid, wherein the cell has been treated or genetically modified to overexpress or express with upregulated activity one or more enzymes selected from:
(a) phosphoglucoisomerase;
(b) D-fructose-6-phosphate amidotransferase;
(c) phosphoglucosamine mutase;
(d) glucosamine-1-phosphate acetyl transferase;
(e) N-acetylglucosamine-1-phosphate pyrophosphorylase
(f) glucosamine-6-phosphate acetyl transferase; and
(g) phosphoacetylglucosamine mutase.

32. A Streptococcus cell comprising the enzymes for synthesis of hyaluronic acid, wherein the cell has been treated or genetically modified to underexpress or not express or express with downregulated activity one or more enzymes selected from:
(a) UDP-N-acetylglucosamine 1-carboxyvinyltransferase; and
(b) undecaprenyldiphospho-muramoylpentapeptide beta-N-acetylglucosaminyltransferase.

33. The cell according to claim 32, wherein at least one copy of a gene encoding UDP-N-acetylglucosamine 1-carboxyvinyltransferase in the cell has been mutated to underexpress or not express or express with downregulated activity UDP-N-acetylglucosa mine 1-carboxyvinyltransferase.

34. A pharmaceutical composition comprising the hyaluronic acid according to claim 28 and a pharmaceutically acceptable carrier, excipient or diluent.

35. A cosmetic composition comprising the hyaluronic acid according to claim 28 and a cosmetically acceptable carrier, excipient or diluent.

36. A food product or food additive comprising the hyaluronic acid according to claim 28.
